(19) United States
(12) Patent Application Publication
(10) Pub. No.: US 2018/0166161 A1
(43) Pub. Date: Jun. 14, 2018

(54) A WORKFLOW MANAGEMENT SYSTEM AND RELATED METHOD FOR MULTI-AUTHOR REPORTING

(71) Applicant: Agfa HealthCare NV, Mortsel (BE)

(72) Inventors: Nancy SAMI, Mortsel (BE); Wim GEERAERTS, Mortsel (BE); Olivier DEBELS, Mortsel (BE); Caroline BOGAERT, Mortsel (BE)

(73) Assignee: Agfa HealthCare NV, Mortsel (BE)

(21) Appl. No.: 15/571,088

(22) PCT Filed: May 3, 2016
(86) PCT No.: PCT/EP2016/059819

§ 371 Date: Nov. 1, 2017

(30) Foreign Application Priority Data
May 7, 2015 (EP) 15166725.0

(51) Int. Cl. G16H 15/00; G06Q 10/06; G06F 17/30

(52) U.S. Cl. CPC G16H 15/00; G06F 17/30554; G06Q 10/06311; G06Q 10/0633

(57) ABSTRACT

A workflow management system for medical report production includes a workflow database that stores workflows, a user database that stores users, a task configurator that generates and configures tasks for production of a medical report in correspondence with rules of a workflow out of the workflows and assigns the tasks to users out of the user database, the tasks including at least one multi-author task, and an exclusion list manager that maintains an exclusion list of excluded authors for the multi-author task, wherein the task configurator is operationally coupled to the exclusion list manager and adapted to exclude the excluded authors when assigning the multi-author task to users.

### CROSS REFERENCE TO RELATED APPLICATIONS

This application is a 371 National Stage Application of PCT/EP2016/059819, filed May 3, 2016. This application claims the benefit of European Application No. 15166725.0, filed May 7, 2015, which is incorporated by reference herein in its entirety.

### BACKGROUND OF THE INVENTION

### 1. Field of the Invention

The present invention generally relates to the production of a medical report, where two or more authors are involved in the creation and the approval of this medical report.

### 2. Description of the Related Art

In a multi-author workflow, two or more authors are involved in the creation and the approval of a medical report. Most multi-author workflows improve the quality of the resulting medical report, since it has been established in the context of medical imaging that the error rate in the interpretation of medical images can be reduced when involving two or more authors in the reporting process. Examples of multi-authors workflows may be: workflows for positron emission tomography-computed tomography, also referred to as PET-CT workflows where a radiologist and a nuclear medicine physician are both involved in creating the medical report; workflows for cardiac computed tomography, also referred to as cardiac CT workflows, where a cardiologist reports on all cardiac investigations and a radiologist reports on all other investigations, such as investigations on lungs, mediastinum, spine, etc.

EP1926041A1 is a patent application from Quadrat published on May 28, 2008, entitled “Configurable workflow and task management method”. The patent application describes a workflow and task management system to steer the workflow of a user in an information system used in healthcare environment. Each workflow participant gets a task list that is tailored to his/her/its responsibilities. The assignment of task instances to a responsible is done using assignment rules which can be pre-filtered. The user can for example specify one or more filters on his task instances, or the task assignment happens more fine-grained, for example, only users which log in as interested in “reading CR of patients which are treated by a certain doctor” will receive the task instances. However, EP1926041A1 does not describe a task management system involving a plurality of authors in the production of a medical report. There exists a risk that, when the responsible belongs to a group to which the task is assigned, the task remains in the task list of the responsible even if the responsible performs the task. Tasks that were finalized by the responsible and tasks to be finalized by the responsible may then co-exist in the task list of the responsible. This creates confusion and consequently jeopardizes the efficiency of the task assignment of the task management system.

U.S. Pat. No. 6,725,428B1 is a patent from Xerox Corporation published on Apr. 20, 2004, entitled “Systems and methods providing flexible representations of work”. The patent describes workflow management techniques for coordinating organizational processes when a plurality of users are involved in the production of a decomposable document. A first workflow management technique consists of mapping each activity one-to-one onto independent sections of the document. In this scenario, various streams of the document exist in parallel and may evolve independently. Additionally, all the co-authors of the document are considered to be peers and their inputs are of the same importance for the generation of the document. In a medical context, the workflow management technique of U.S. Pat. No. 6,725,428B1 would limit the quality of a medical report. Indeed, some authors may require to access the conclusions of other authors before being able to report their own interpretation of the medical data.

U.S. Pat. No. 6,725,428B1 further describes a second workflow management technique used for the production of a non-decomposable document by a plurality of users. The second workflow management technique consists of coordinating concurrent authoring and editing of the document. In this scenario, independent subdocuments exist and may evolve independently. The document is created after collecting and merging several independent subdocuments, where the merging relies on a grammar of rules to ensure the quality of the operation. The method described in U.S. Pat. No. 6,725,428B1 is therefore complex, and its complexity reduces the efficiency of the production of the document and results in a slow throughput for the generation of a multi-author report. Furthermore, there exists a risk that independent subdocuments are overlooked or overwritten during the merging. This jeopardizes the quality of the resulting document, and is unacceptable in a medical context.

### SUMMARY OF THE INVENTION

Preferred embodiments of the present invention disclose a system and the related method that overcome the above identified shortcomings of existing solutions. More particularly, preferred embodiments of the present invention disclose such a workflow management system and method that are less error-prone and that improve the experience of a co-authors of a medical report by reducing the amount of already completed or performed tasks from the task list of co-authors. Further preferred embodiments disclose such a system and method that increase the flexibility for an author to request review of the medical report by co-authors.

According to a first aspect of the present invention, the above defined objectives are realized by a workflow management system for medical report production, the workflow management system comprising:
a workflow database, configured to store workflows;
a user database, configured to store users; and
a task configurator, operationally coupled to the workflow database and the user database, and adapted to generate and configure tasks for production of a medical report in correspondence with rules of a workflow out of the workflows, and adapted to assign the tasks to users out of the user database, the tasks comprising at least one multi-author task;
   CHARACTERIZED IN THAT the workflow management system further comprises:
an exclusion list manager, configured to maintain an exclusion list of excluded authors for the multi-author task; and further in that:
the task configurator is operationally coupled to the exclusion list manager and adapted to exclude the excluded authors when assigning the multi-author task to users.

In accordance with a preferred embodiment of the present invention, a user database is adapted to store users. Users are entities to whom one or more tasks, in particular one or more multi-author tasks, may be assigned. A user stored in the user database may be an individual or may be an assignment group comprising more than one individual. A multi-author task is a task generated and configured by the task configurator and assigned to more than one individual, for example two, three, four, five, etc. individuals and/or one or more assignment groups, for example two, three, four, etc. An author of a multi-author task is an individual of the individuals to whom the multi-author task is assigned who performs the multi-author task or an author of a multi-author task is an individual from an assignment group to whom the multi-author task is assigned who performs the multi-author task.

According to a preferred embodiment of the present invention, an author is therefore a user of the workflow management system taking part in the production of a medical report. For example, an author is responsible for the production of one or more sections of a medical report. Workflows are configured using rules. The rules result in the creation of tasks. The number of created tasks is equal to the number of authors for which the workflow is configured. Tasks are automatically visible in the task list of the appropriate user, and the task list lists all the tasks a user is assigned in order to guide the user into his daily job. The purpose of a task is the creation of a medical report. The tasks are not necessarily all created at the same time.

The workflow management system according to preferred embodiments of the present invention is less prone to errors. Indeed, a task is only created when the workflow is initiated or when an author completes or performs one of his tasks. The risk that an author works on a medical report before all input from co-authors is available is therefore reduced. Additionally, tasks performed by an author of the workflow management system are deleted from the task list of the author. The user is added to an exclusion list for the corresponding multi-author task, and is therefore considered as an excluded author for the corresponding multi-author task. This way, only tasks that still need to be performed by the author are visible in the task list of the author. This reduces the risk that a task is executed twice by the same author or by two different authors, and therefore reduces the chance that an error impacts the production of a medical report. This also considerably improves the efficiency of the management of tasks in the workflow management system and thereby improves the throughput of the users. Additionally, the workflow management system does not require the creation of several independent subdocuments, where each subdocument would be completed by an author and where all the independent subdocuments would be merged in order to produce the medical report. The workflow management system therefore reduces the risk of overlooking and/or overwriting sections of the medical report.

The exclusion list manager updates exclusions lists associated to corresponding multi-author tasks, thereby adding and/or removing excluded authors to/from the exclusion lists. In accordance with a preferred embodiment of the present invention, to each multi-author task corresponds one exclusion list. According to an alternative embodiment, to each multi-author task may be associated a plurality of exclusions lists, for example an exclusion list for individual authors and an exclusion list for assignment groups. According to a further alternative embodiment, an exclusion list may be associated to a plurality of multi-author tasks. An exclusion list may be empty when the workflow is initiated. Alternatively, an exclusion list may already comprise one or more excluded authors when the workflow is initiated. Adding an excluded author to an exclusion list prevents the assignment of one or more multi-author tasks corresponding to the exclusion lists to the excluded author. On the contrary, removing an excluded author from an exclusion list allows the assignment of one or more multi-author tasks corresponding to the exclusion list to the excluded author.

In other words, depending on the configuration of the rules of the workflow, users that are identified as authors having completed a multi-author task for the production of a medical report are added to the exclusion list for the multi-author task and will not receive subsequent reading tasks for the same medical report in their respective task lists anymore. Also, individuals belonging to the same assignment group as the author having completed the multi-author task are added to the exclusion list for that medical report and will not receive tasks for the same medical report in their respective task lists.

According to an optional embodiment, a user is one of the following:
an individual; or
an assignment group comprising more than one individuals;
   and the multi-author task is a task assigned to at least plural individuals and/or one or more assignment groups.

A user in the user database may be an individual user of the workflow management system, and/or may be an assignment group comprising more than one individual users of the workflow management system. An individual may belong to one or more assignment groups. A multi-author task may consequently be assigned to one or more individuals, for example two, three, four, five individuals and/or one or more assignment groups. An author of a multi-author task is an individual of the individuals to whom the multi-author task is assigned who performs the multi-author task or an author of a multi-author task is an individual from an assignment group to whom the multi-author task is assigned who performs the multi-author task.

According to an optional embodiment, the exclusion list manager is configured to:
add an assignment group to the exclusion list when an individual that forms part of the assignment group performs the multi-author task;
add an individual that forms part of an assignment group to the exclusion list when the individual performs the multi-author task with a request for review by co-authors of the assignment group; and
remove a user from the exclusion list when an individual performs the multi-author task with a request for review by the user listed in the exclusion list.

Several possible different scenarios may unfold depending on the performance of the multi-author task by an individual. An individual performs a multi-author task when the individual creates, and/or edits, and/or partially validates a medical report that was created, and/or edited, and/or partially validated by the individual, and/or by another individual. A multi-author task ends when one of the individuals fully validates the medical report. When the multi-author task is performed by an individual forming part of an assignment group, the assignment group to which the individual belongs is added to the exclusion list corresponding to the multi-author task. This way, once the assignment group of the individual is added to the exclusion list for the multi-author task, the multi-author task is not visible in the task list of the individuals of the assignment group anymore. The risk that the multi-author task is performed again by another individual of the same assignment group is therefore limited, and the workflow management system is then less prone to errors. When the multi-author task is performed by an individual forming part of an assignment group and the individual requests a second opinion, i.e. a review by a co-author of the assignment group, the individual is added to the exclusion list for the multi-author task. This way, the multi-author task is not visible in the task list of the individual anymore, and the risk that the individual performs the same multi-author task twice is lowered. The task however remains visible in the task lists of other individuals of the assignment group. The efficiency of the management system is therefore improved. When a multi-author task is performed by an individual with a request for review by a user listed in the exclusion list, the user is removed from the exclusion list for the multi-author task. This way, the multi-author task is added to the task list of the user. A user may be an individual or an assignment group comprising more than one individuals. This way, the workflow management system improves the experience of the users of the user database by increasing the flexibility of the management of tasks and by offering the possibility to authors to request co-authors to review the medical report. This way, a second opinion on the contribution of one or more authors can be asked to co-authors of the medical report and/or to more qualified or more experienced users. The implementation of such possible feedback loops improves the overall quality of the medical report.

According to an optional embodiment, the task configurator is further configured to:
assign the multi-author task to users in correspondence with the rules of the workflow;
receive the exclusion list for the multi-author task from the exclusion list manager; and
filter the users whereto the multi-author task is assigned using the exclusion list, thereby preventing assigning the multi-author task to authors listed in the exclusion list.

Preferably, the assignment of the tasks is performed at different levels of assignment from which a final assignment is extracted. In a first level of assignment, the multi-author task is assigned to users in correspondence with the rules of the workflow. Subsequently, in a second level of assignment, the assignment of the multi-author task resulting from the first level of assignment is filtered in accordance with the exclusion list for the multi-author task. The second level of assignment therefore removes excluded authors from the list of users to whom the multi-author task was assigned after the first level of assignment. The result of the second level of assignment is the final assignment of the multi-author task. In other words, the multi-author task is not assigned to excluded authors listed in the exclusion list. The multi-author task is only visible in the task list of users to whom the multi-author task is assigned after the first level of assignment and who are not listed in the exclusion list for the multi-author task.

According to an optional embodiment, the task configurator is further adapted to subsequently assign the multi-author task whenever the multi-author task is performed by an author.

This way, when an author performs a multi-author task, the task configurator subsequently assigns the multi-author task to an individual and/or an assignment group based on the performance of the multi-author task and/or based on the rules of the workflow. The assignment of the tasks may be triggered automatically or may be triggered manually.

According to a second aspect of the invention, there is provided an exclusion list manager for use in a workflow management system as defined according to a first aspect of the invention, the exclusion list manager comprising:
an exclusion list generator, adapted to generate an exclusion list of excluded authors for a multi-author task;
an exclusion list updater, configured to:
   add an assignment group to the exclusion list when an individual that forms part of the assignment group performs the multi-author task;
   add an individual that forms part of an assignment group to the exclusion list when the individual performs the multi-author task with a request for review by users of the assignment group; and
   remove a user from the exclusion list when an individual performs the multi-author task with a request for review by the user listed in the exclusion list.

An individual may be an individual or may be an individual forming part of an assignment group comprising more than one individual. A multi-author task may be assigned to more than one individuals, for example two, three, four, five, etc. individuals and/or one or more assignment groups comprising more than one individuals. An author of a multi-author task is an individual of the individuals to whom the multi-author task is assigned who performs the multi-author task or an author of a multi-author task is an individual from an assignment group to whom the multi-author task is assigned who performs the multi-author task. Tasks are automatically visible in the task list of the appropriate user, and the task list lists all the tasks a user is assigned in order to guide the user into his daily job. The purpose of a task is the creation of a medical report.

The exclusion list manager generates an exclusion list of excluded authors for a multi-author task. The exclusion list manager further updates the exclusions list associated with a corresponding multi-author task depending on actions of an author, thereby adding and/or removing excluded authors to/from the exclusion list. Several possible different scenarios may unfold depending on the performance of the multi-author task. When a multi-author task is performed by an individual forming part of an assignment group, the assignment group to which the individual belongs is added to the exclusion list corresponding to the multi-author task. This way, once the assignment group of the individual is added to the exclusion list for the multi-author task, the multi-author task is not visible in the task list of the individuals of the assignment group anymore. The risk that the multi-author task is performed again by another individual of the same assignment group is therefore limited, and the workflow management system is then less prone to errors. When the multi-author task is performed by an individual forming part of an assignment group and the individual requests a second opinion, i.e. a review by a co-author of the assignment group, the individual is added to the exclusion list for the multi-author task. This way, the multi-author task is not visible in the task list of the individual anymore, and the risk that the individual performs the same multi-author task twice or that the multi-author task is overwritten is lowered. The efficiency of the management system is therefore improved. When a multi-author task is performed by an individual with a request for review by a user listed in the exclusion list, the user is removed from the exclusion list for the multi-author task. A user may be an individual or an assignment group comprising more than one individual. This way, the multi-author task is visible in the task list of the user. The workflow management system further improves the experience of the users as the amount of tasks in the tasks lists is limited to tasks that still need to be performed. This way, the workflow management system improves the experience and the throughput of the users of the user database by increasing the flexibility of the management of tasks and by offering the possibility to authors to request co-authors to review the medical report. A second opinion on the contribution of one or more authors can be asked to co-authors of the medical report and/or to more qualified and/or more experienced users. The implementation of such possible feedback loops improves the overall quality of the medical report.

To each multi-author task is associated one exclusion list. According to an alternative embodiment, to each multi-author task may be associated a plurality of exclusions lists, for example an exclusion list for individuals and an exclusion list for assignment groups. According to a further alternative embodiment, an exclusion list may be associated to a plurality of multi-author tasks. An exclusion list may be empty when the workflow is initiated. Alternatively, an exclusion list may already comprise one or more excluded authors when the workflow is initiated. Adding an excluded author to an exclusion list prevents the assignment of one or more multi-author tasks corresponding to the exclusion lists to the excluded author. On the contrary, removing an excluded author from an exclusion list allows the assignment of one or more multi-author tasks corresponding to the exclusion list to the excluded author.

According to a third aspect of the invention, there is provided a method for managing exclusion lists in a workflow management system as defined according to a first aspect of the invention, the method comprising the steps of:
generating an exclusion list of excluded authors for a multi-author task;
updating the exclusion list by:
   adding an assignment group to the exclusion list when an individual that forms part of the assignment group performs the multi-author task;
   adding an individual that forms part of an assignment group to the exclusion list when the individual performs the multi-author task with a request for review by users of the assignment group; and
   removing a user from the exclusion list when an individual performs the multi-author task with a request for review by the user listed in the exclusion list.

According to a fourth aspect of the invention, there is provided a task configurator for use in a workflow management system as defined according to a first aspect of the invention, the task configurator comprising:
a workflow interface, adapted to receive a workflow from a workflow database;
a user interface, adapted to receive users from a user database;
a task configuring unit, adapted to configure tasks for production of a medical report in correspondence with rules of the workflow, the tasks comprising at least one multi-author task;
a task assigning unit, adapted to assign the multi-author task to users in correspondence with rules of the workflow;
an exclusion list interface, adapted to receive an exclusion list of excluded authors for the multi-author task;
a task filtering unit, adapted to filter the users whereto the multi-author task is assigned using the exclusion list, thereby preventing assigning the multi-author task to the excluded authors.

According to a fifth aspect of the invention, there is provided a method for configuring tasks in a workflow management system as defined according to a first aspect of the invention, the method comprising the steps of:
receiving a workflow from a workflow database;
receiving users from a user database;
configuring tasks for production of a medical report in correspondence with rules of the workflow, the tasks comprising at least one multi-author task;
assigning the multi-author task to users in correspondence with the rules of the workflow;
receiving an exclusion list of excluded authors for the multi-author task;
filtering the users whereto the multi-author task is assigned using the exclusion list, thereby preventing assigning the multi-author task to the excluded authors.

### BRIEF DESCRIPTION OF THE DRAWINGS

FIG. 1 schematically illustrates an embodiment of a workflow management system for the production of a medical report.

FIG. 2 schematically illustrates an embodiment of an exclusion list manager for use in the workflow management system of FIG. 1.

FIG. 3 schematically illustrates an embodiment of a task configurator for use in the workflow management system of FIG. 1.

FIG. 4 schematically illustrates a diagram of chronological actions performed in the workflow management system of FIG. 1.

FIG. 5 schematically illustrates the steps of a method for generating and updating an exclusion list of excluded authors for a multi-author task.

FIG. 6 schematically illustrates the steps of a method for configuring and assigning tasks comprising at least one multi-author task, and for filtering excluded authors from the assignment of the multi-author task.

### DETAILED DESCRIPTION OF THE PREFERRED EMBODIMENTS

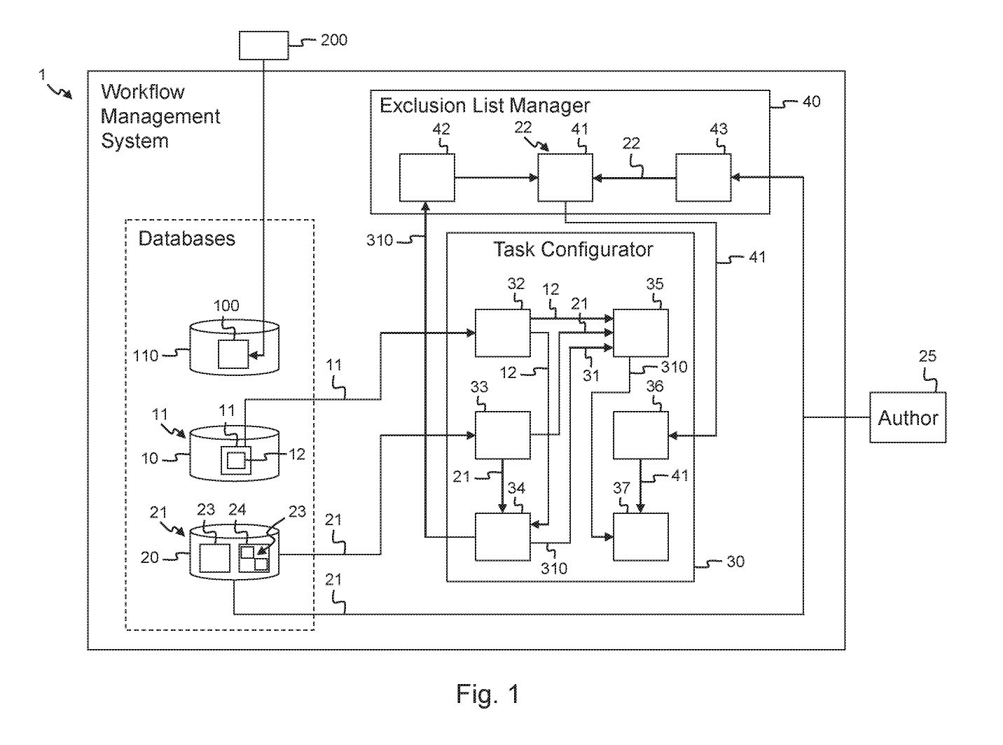

According to an embodiment shown in FIG. 1, the workflow management system 1 comprises a workflow database 10, a user database 20, a task configurator 30 and an exclusion list manager 40. The workflow database 10 is adapted to store workflows 11. As visible in FIG. 1, the workflow database 10 stores one workflow 11. According to alternative embodiments, the workflow database 10 is adapted to store a plurality of workflows 11. Each workflow 11 comprises rules 12 resulting in the creation of tasks 31. The user database 20 is adapted to store users 21. Users 21 of the workflow management system 1 may be individuals 23 or assignment groups 24. The user database 20 is adapted to store one or more individuals 23 and/or one or more assignment groups 24. An assignment group 24 may comprise two or more individuals 23. The task configurator 30 comprises a workflow interface 32, a user interface 33, a task configuring unit 34, a task assigning unit 35, an exclusion list interface 36 and a task filtering unit 37. The workflow interface 32 receives a workflow 11 that is initiated. The user interface 33 receives users 21 from the user database 20. The task configuring unit 34 configures tasks 31 for production of a medical report 100 in correspondence with the rules 12 of the workflow 11. The tasks 31 comprise one multi-author task 310. According to an alternative embodiment, the tasks 31 comprise one or more multi-author tasks 310. The task assigning unit 35 assigns the multi-author task 301 to users 21 in correspondence with the rules 12 of the workflow 11. The task assigning unit 35 further assigns the tasks 31 to users 21 in correspondence with the rules 12 of the workflow 11. The exclusion list interface 36 receives an exclusion list 41 of excluded authors 22 for the multi-author task 310. According to an alternative embodiment, the exclusion list interface 36 receives a plurality of exclusion lists 41. The task filtering unit 37 filters the users 21 whereto the multi-author task is assigned using the exclusion list 41, and thereby prevents the assignment of the multi-author task 310 to users 21 comprised in the exclusion list 41, i.e. to excluded authors 22 for the multi-author task 310. The exclusion list manager 40 comprises an exclusion list generator 42 and an exclusion list updater 43. The exclusion list generator 42 generates an exclusion list 41 of excluded authors 22 for the multi-author task 310. According to an alternative embodiment, the exclusion list generator 42 generates one or more exclusion lists 41 of excluded authors 22 for the multi-author task 310, for example one exclusion list 41 comprising individuals 23 and one exclusion list 41 comprising assignment groups 24. The exclusion list updater 43 updates the exclusion list 41. An author 25 of a medical report interacts with the workflow management system 1. In the case where an individual 23 is an author 25 of the multi-author task 310, and when the author 25 forms part of an assignment group 24 and when the author 25 performs the multi-author task 310, then the exclusion list updater 43 adds the entire assignment group 24 of the author 25 to the excluded authors 22 of the exclusion list 41 for the multi-author task 310. In the case where an individual 23 is an author 25 of the multi-author task 310, and when the author 25 forms part of an assignment group 24 and when the author 25 performs the multi-author task 310 with a request for review by one or more users 21 of the assignment group 24, then the exclusion list updater 43 adds the author 25 to the excluded authors 22 of the exclusion list 41 for the multi-author task 310. In the case where an individual 23 is an author 25 of the multi-author task 310, and when the author 25 performs the multi-author task 310 with a request for review by one or more users 21 listed in the exclusion list 41 for the multi-author task 310, the exclusion list updater 43 removes the one or more users 21 from the exclusion list 41 of excluded authors 22 for the multi-author task 310. The multi-author task 310 is communicated by the task configuring unit 34 of the task configurator 30 to the exclusion list generator 42. The exclusion list 41 is communicated by the exclusion list manager 40 to the exclusion list interface 36 of the task configurator 30. According to alternative embodiments, the task configurator 30 and the exclusion list manager 40 are operationally coupled to each other. According to an optional embodiment, the workflow management system 1 further comprises a medical report database 110, adapted to store one or more medical reports 100. A workflow 11 is initiated when medical data 200 is received in the workflow management system 1, or when an author 25 manually initiates the workflow 11. The produced medical report 100 resulting from the completion of tasks 31 and/or of the multi-author task 310 is stored in the medical report database 110. In a possible embodiment of the current invention, only one initial task 31 is created when the workflow 11 is initiated. When a first author 25 performs the initial task 31, i.e. partially validates the medical report 100, a second task 31 may be created. According to an alternative embodiment, the first author may fully validate the multi-author task 310 when performing the initial task 31. When the medical report 100 is partially signed or partially validated after the second task is performed, a third task 31 may be created, etc. According to an alternative embodiment, the multi-author task 310 may be fully validated when performing the second task. The workflow 11 ends when a user 21 of the workflow management system 1 fully signs off the medical report. For example, the workflow 11 may end when the user 21 to which the last task 31 of the workflow 11 was assigned fully signs the medical report 100 off.

For example, the task configurator 30 may have to configure a multi-author task 310 for users 21 with the same medical specialism and according to rules 12 of a workflow 11. In the case where a multi-author task 310 exists and is linked to a modality type “mammography” and there exists no medical report 100 yet, then the task configurator 30 assigns the multi-author task 310 to the assignment group 24 “radiologists”. This defines the assignment of the initial multi-author task 310 when no medical report 100 is available. Alternatively, in the case where a multi-author task 310 exists and is linked to the modality type “mammography” and the multi-author task 310 is performed, the task configurator 30 assigns the multi-author task 310 to the assignment group 24 “radiologists” but not to the previous author 25 or authors 25 of the medical report 100. This rule 12 defines the subsequent assignment of the multi-author task 310, when one or more previous authors 25 has/have performed the multi-authors task 310. In other words, the previous author 25 or authors 25 that performed the multi-author task 310 for the same medical report 100 is/are added to the exclusion list 41 of excluded authors 22 for the multi-author task 310. In other words, the multi-author task 310 is assigned to the assignment group 24 “radiologists” but the radiologists who performed the multi-author task 310 will no longer see the multi-author task 310 in their respective task lists.

For example, the task configurator 30 may have to configure a multi-author task 310 for users 21 with different medical specialism and according to rules 12 of a workflow 11. In the case where a multi-author task 310 exists and is linked to a procedure definition “Cardiac CT” and there exists no medical report 100 yet, then the task configurator 30 assigns the multi-author task 310 to the assignment group 24 “radiologists” and to the individual 23 who is a particular cardiologist of the assignment group 24. This defines the assignment of the initial multi-author task 310 when no medical report 100 is available. Alternatively, in the case where a multi-author task 310 exists and is linked to the procedure definition “Cardiac CT” and the multi-author task 310 is performed, the task configurator 30 assigns the multi-author task 310 to the assignment group 24 “radiologists” and to the individual 23 who is a particular cardiologist in the assignment group 24 but the task configurator 30 will not assign the multi-author task 310 to the assignment groups 24 of the previous one or more authors 25 of the medical report 100 and also not to the previous one or more authors 25 of the medical report 100. In other words, the previous author 25 or authors 25 that performed the multi-author task 310 and his/their assignment groups 24 are added to the exclusion list 41 of excluded authors 22 for the multi-author task 310. In other words, if the initial assignment of the multi-author task 310 is first performed by an individual 23, the subsequent assignment of the multi-author task 310 will be directed to all the radiologists and one of the radiologists will perform the multi-author task 310. On the contrary, if the initial assignment is performed by one of the radiologists, the subsequent assignment of the multi-author task 310 will be directed to the individual 23 only.

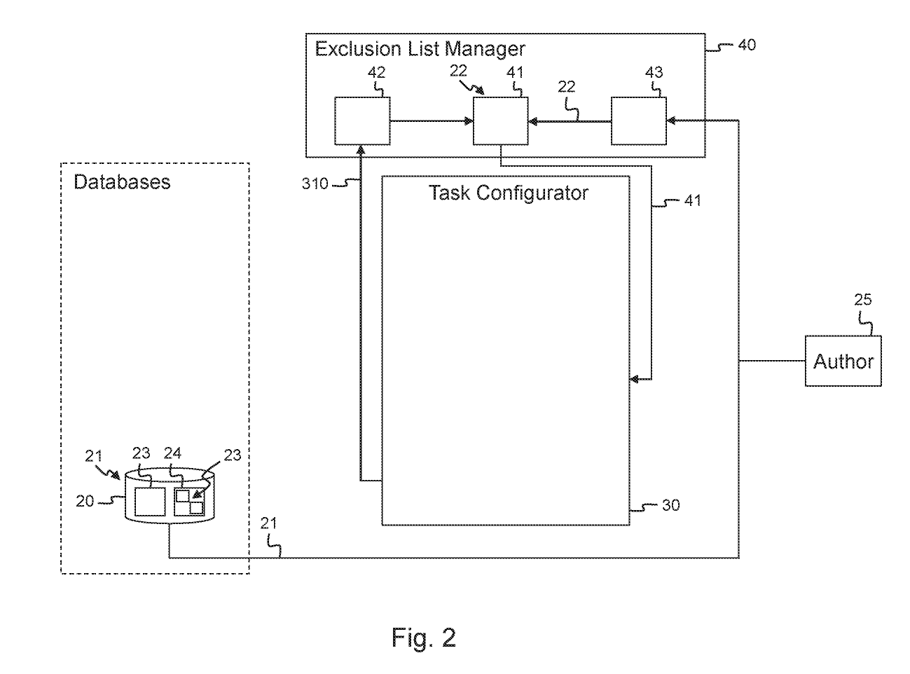

According to an embodiment shown in FIG. 2, the exclusion list manager 40 for use in a workflow management system 1 as described in FIG. 1 comprises an exclusion list generator 42 and an exclusion list updater 43. The exclusion list generator 42 generates an exclusion list 41 of excluded authors 22 for the multi-author task 310. According to an alternative embodiment, the exclusion list generator 42 generates one or more exclusion lists 41 of excluded authors 22 for the multi-author task 310, for example one exclusion list 41 comprising individuals 23 and one exclusion list 41 comprising assignment groups 24. The exclusion list updater 43 updates the exclusion list 41. An author 25 of a medical report interacts with the workflow management system 1. In the case where an individual 23 is an author 25 of the multi-author task 310, and when the author 25 forms part of an assignment group 24 and when the author 25 performs the multi-author task 310, then the exclusion list updater 43 adds the entire assignment group 24 of the author 25 to the excluded authors 22 of the exclusion list 41 for the multi-author task 310. In the case where an individual 23 is an author 25 of the multi-author task 310, and when the author 25 forms part of an assignment group 24 and when the author 25 performs the multi-author task 310 with a request for review by users 21 of the assignment group 24, then the exclusion list updater 43 adds the author 25 to the excluded authors 22 of the exclusion list 41 for the multi-author task 310. In the case where an individual 23 is an author 25 of the multi-author task 310, and when the author 25 performs the multi-author task 310 with a request for review by users 21 in the exclusion list 41, the exclusion list updater 43 removes the users 21 from the exclusion list 41 of excluded authors 22 for the multi-author task 310. The multi-author task 310 is communicated by the task configurator 30 to the exclusion list generator 42. The exclusion list 41 is communicated by the exclusion list manager 40 to the task configurator 30. The task configurator 30 and the exclusion list manager 40 are operationally coupled to each other.

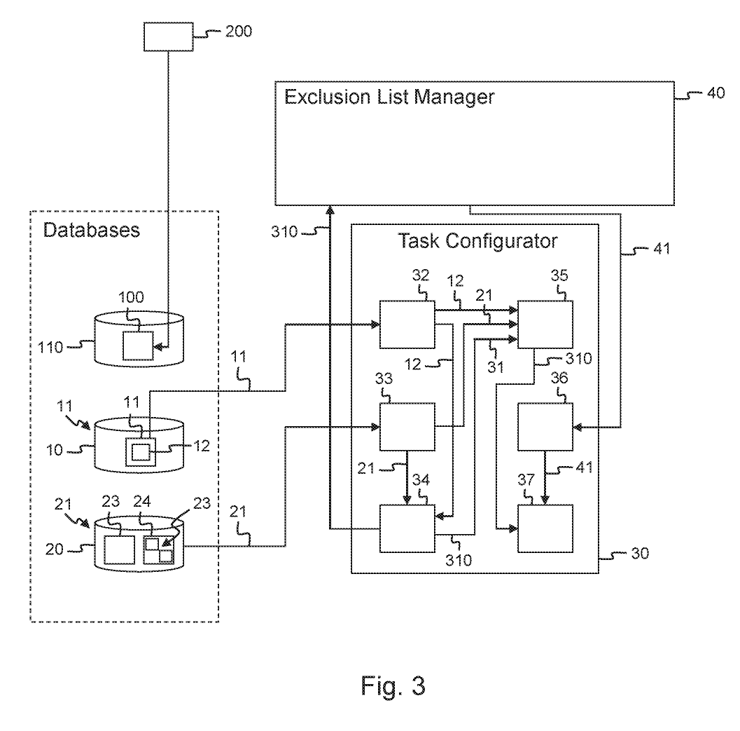

According to an embodiment shown in FIG. 3, the task configurator 30 for use in a workflow management system 1 as described in FIG. 1 comprises a workflow interface 32, a user interface 33, a task configuring unit 34, a task assigning unit 35, an exclusion list interface 36 and a task filtering unit 37. The workflow interface 32 receives a workflow 11 that is initiated. The user interface 33 receives users 21 from the user database 20. The task configuring unit 34 configures tasks 31 for production of a medical report 100 in correspondence with the rules 12 of the workflow 11. The tasks 31 comprise one multi-author task 310. According to an alternative embodiment, the tasks 31 comprise one or more multi-author task 310. The task assigning unit 35 assigns the multi-author task 301 to users 21 in correspondence with the rules 12 of the workflow 11. The task assigning unit 35 further assigns the tasks 31 to users 21 in correspondence with the rules 12 of the workflow 11. The exclusion list interface 36 receives an exclusion list 41 of excluded authors 22 for the multi-author task 310. According to an alternative embodiment, the exclusion list interface 36 receives a plurality of exclusion lists 41. The task filtering unit 37 filters the users 21 whereto the multi-author task is assigned using the exclusion list 41, and thereby prevents the assignment of the multi-author task 310 to users 21 comprised in the exclusion list 41, i.e. to excluded authors 22. The multi-author task 310 is communicated by the task configurator 30 to the exclusion list generator 42. The exclusion list 41 is communicated by the exclusion list manager 40 to the task configurator 30. The task configurator 30 and the exclusion list manager 40 are operationally coupled to each other.

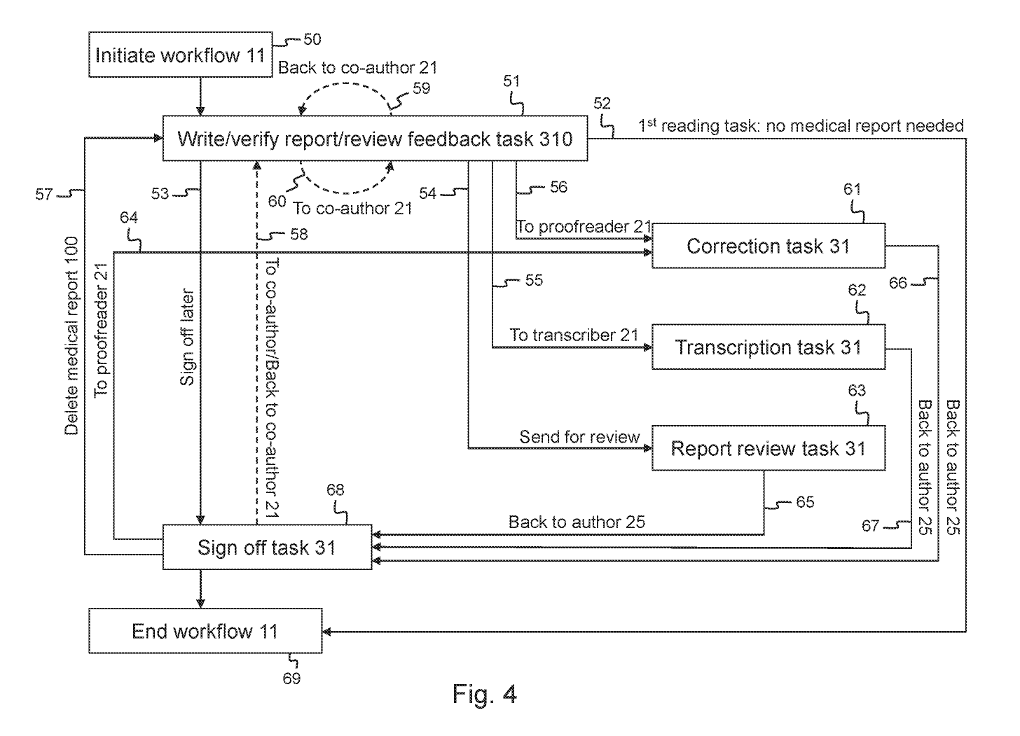

According to an embodiment shown in FIG. 4, a workflow 11 is first initiated at the step 50 in order to produce a medical report 100. A multi-author task 310 of the workflow 11 is then performed at step 51 by an individual 23 of the user database 20 that may or may not belong to an assignment group 24 for the multi-author task 310 and to whom the multi-author task 310 is assigned. This individual 23 performing the multi-author task 310 is then further referred to as the author 25 of the multi-author task 310. The multi-author task 310 may consist in writing a section of the medical report 100, in verifying and/or completing one or more sections of the medical report 100, and/or in requesting and/or providing review and feedback over one or more sections of the medical report 100. Step 51 may therefore consist in writing a medical report 100, and/or in verifying and/or completing a medical report 100, and/or in requesting or reviewing a medical report 100. In other words, step 51 is related to a multi-author task 310 for which an exclusion list 41 of excluded authors 22 for the multi-author task 310 is used when assigning the multi-author task 310. This step 51 may for example result in a new assignment for which the medical report 100 is sent to a co-author 21 for review in step 60. Step 51 may also for example result in a new assignment for which the medical report 100 is sent back to a co-author 21 in step 59. In the case 52 where no medical report 100 must be created, the multi-author task 310 is cancelled. This ends the workflow 11 in step 69. In the case 54 where the author 25 performs the multi-author task 310, another user 21 of the database 20 may perform a review of the medical report 100 in step 63. The other user 21 of the user database 20 may then perform a review task and the other user 21 sends the medical report 100 back to the author 25, who is assigned a sign off task 31 in step 68 that ends the workflow 11 in step 69. In the case 55 where the author 25 performs the multi-author task 310, a transcriber 21, i.e. another user of the database 20, may perform a transcription task in step 62. For example, the author 25 may dictate and record his performance of the multi-author task 310. As a consequence, the performance of the multi-author task 310 by the author 25 may be transcribed in step 62. The transcriber 21 sends the medical report back to the author, who performs a sign off task 31 in step 68 which ends the workflow 11 in step 69. In the case 56 where the author 25 performs the multi-author task 310, another user 21 of the user database 20 may proofread the medical report 100. The other user 21 may perform a correction task 31 in step 61. The other user 21 sends the medical report back to the author, who performs a sign off task 31 which ends the workflow 11 in step 69. In any of the cases 65, 66, 67, the author 25 and/or another user 21 of the user database 20 may request in step 68 proofreading of the medical report 100, and the medical report 100 is then sent back to a proofreader 21 in the case 64. A subsequent correction task 31 is performed in step 61 and a sign off task 31 may be performed in step 68 which ends the workflow 11 in step 69. In any cases 52, 53, 65, 66, 67, the author 25 may delete the medical report 100 in the case 57, thereby automatically ending the workflow 11 in step 69. In the case 53 where the author 25 performs the multi-author task 310, the performance of the multi-author task 310 is recorded, for example the section of a medical report 100 written, read and/or corrected is saved in the workflow management system, and the author 25 may decide not to immediately fully or partially validate the multi-author task 310 after its completion. A “sign off later” task is generated in step 53 for the author 25, and this “sign off later” task is visible in a task list of the author 25 different from the task list associated with the multi-author task 310. This way, an author 25 who prefers waiting before signing the multi-author task 310 off is able to save his performance of the multi-author task 310 and to later access this performance to review, modify and/or complete it before signing the multi-author task 310 off. This further improves the quality of the production of the medical report 100 generated by the multi-author task 310.The tasks in the steps 61, 62, and 63 are tasks performed by a single report contributor, such as a transcriber, a corrector, a reviewer, etc., and are independent from the multi-author task 310 from step 51. The exclusion list used for assigning the multi-author task 310 in step 51 is not used when determining the performance of the single author tasks 31 in step 61, 62 or 63.

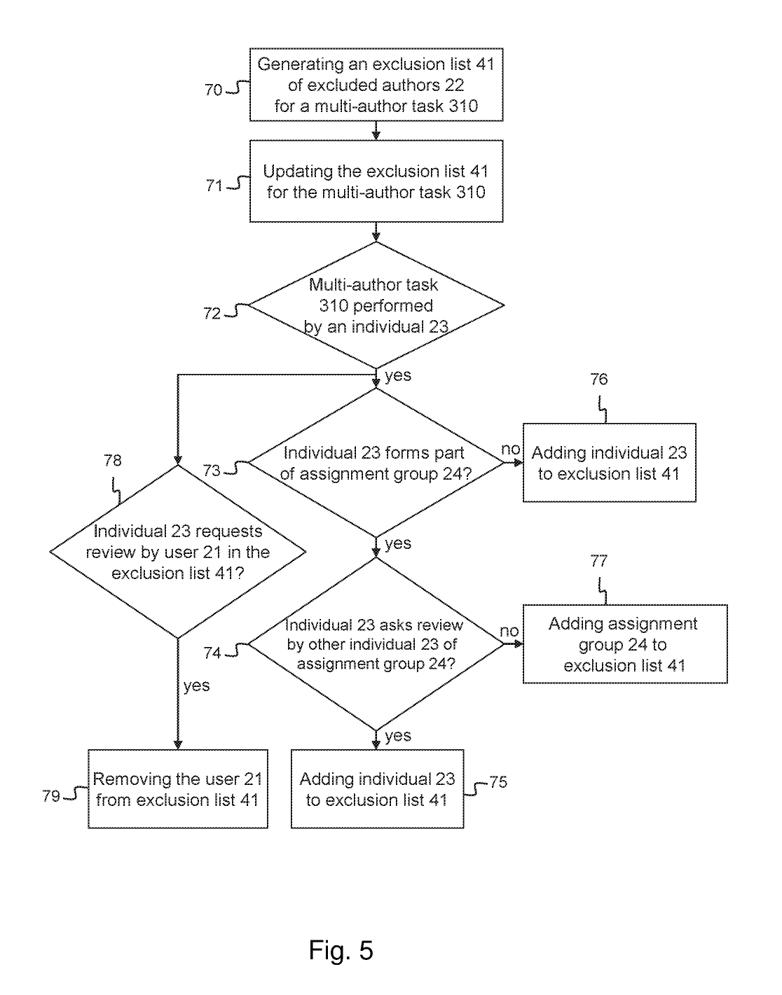

According to an embodiment shown in FIG. 5, an exclusion list 41 of excluded authors 22 for a multi-author task 310 is generated for the corresponding multi-author task 310 in step 70. In step 71, the exclusion list 41 is updated according to several possible scenarios. In step 72, the method comprises the step of checking that the multi-author task 310 is performed by an individual 23. In step 73, the method comprises the step of determining of the individual 23 performing the multi-author task 310 forms part of an assignment group 24. In the case where the individual 23 does not form part of an assignment group 24, the method comprises the step of adding the individual 23 to the exclusion list 41 of excluded authors 22 for the multi-author task 310 in step 76. In the case where the individual 23 does form part of an assignment group 24, the method comprises the step of determining whether the individual 23 requests a review of the medical report 100 by an individual 23 of the assignment group 24 in the step 74. In the case where the author 25 requests a review by an individual 23 of the assignment group 24, the method comprises the step of adding the individual 23 to the exclusion list 41 of excluded authors 22 in step 75. In the case where the author 25 does not request a review by an individual 23 of the assignment group 24, the method comprises the step of adding the assignment group 24 to the exclusion list 41 in step 77, i.e. adding all the individuals 23 forming part of the assignment group 24 to the exclusion list 41 of excluded authors 22 for the multi-author task 310. Alternatively, when the multi-author task 310 is performed in step 72, the method comprises the step of determining if the individual 23 requests a review of the medical report 100 by a user 21 comprised in the exclusion list 41 for the corresponding multi-author task 310 in step 78. If the individual 23 requests a review by a user 21 comprised in the exclusion list 41 for the corresponding multi-author task 310, the method comprises the step of adding the individual 23 to the exclusion list 41 of excluded authors 22 for the multi-author task 310 and of removing the one or more users 21, from whom the individual 23 requested a review, from the exclusion list 41 for the multi-author task 310.

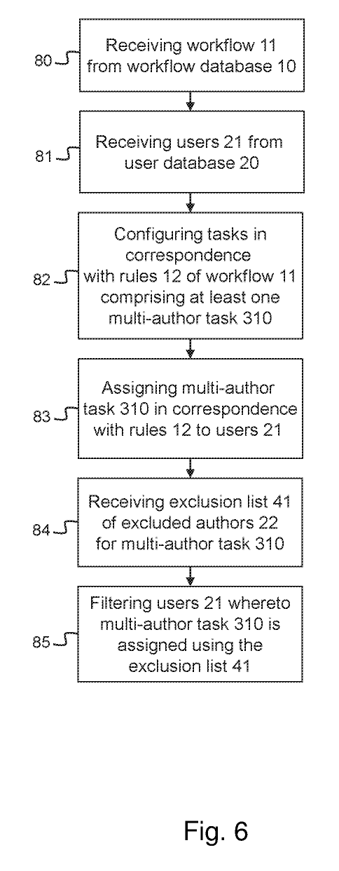

According to an embodiment shown in FIG. 6, the workflow management system 1 receives a workflow 11 from a workflow database 10 in step 80. The workflow management system 1 then receives users 21 from a user database 20 in step 81. The workflow management system 1 subsequently configures tasks 31 in correspondence with the rules 12 of the received workflow 11 in step 82, and the tasks 31 comprise at least one multi-author task 310. In step 83, the workflow management system 1 then assigns the multi-author task 310 to the received users 21 in correspondence with the rules 12 of the received workflow 11. Subsequently, the workflow management system 1 receives an exclusion list 41 of excluded authors 22 for the multi-author task 310 in step 84. Finally, the workflow management system 1 filters users 21 whereto the multi-task 310 is assigned using the exclusion list 41, thereby preventing that the multi-author task 310 is assigned to excluded authors 22 of the exclusion list 41 for the corresponding multi-author task 310.

Although the present invention has been illustrated by reference to specific embodiments, it will be apparent to those skilled in the art that the invention is not limited to the details of the foregoing illustrative embodiments, and that the present invention may be embodied with various changes and modifications without departing from the scope thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein. In other words, it is contemplated to cover any and all modifications, variations or equivalents that fall within the scope of the basic underlying principles and whose essential attributes are claimed in this patent application. It will furthermore be understood by the reader of this patent application that the words “comprising” or “comprise” do not exclude other elements or steps, that the words “a” or “an” do not exclude a plurality, and that a single element, such as a computer system, a processor, or another integrated unit may fulfil the functions of several means recited in the claims. Any reference signs in the claims shall not be construed as limiting the respective claims concerned. The terms “first”, “second”, third”, “a”, “b”, “c”, and the like, when used in the description or in the claims are introduced to distinguish between similar elements or steps and are not necessarily describing a sequential or chronological order. Similarly, the terms “top”, “bottom”, “over”, “under”, and the like are introduced for descriptive purposes and not necessarily to denote relative positions. It is to be understood that the terms so used are interchangeable under appropriate circumstances and embodiments of the invention are capable of operating according to the present invention in other sequences, or in orientations different from the one(s) described or illustrated above.

1-9. (canceled)

10. A workflow management system for producing a medical report, the workflow management system comprising:
a workflow database that stores workflows;
a user database that stores users;
a task configurator, operationally coupled to the workflow database and the user database, that generates and configures tasks to produce a medical report according to rules of one of the workflows stored in the workflow database, and assigns the tasks to users stored in the user database, the tasks including a multi-author task; and
an exclusion list manager that maintains an exclusion list of excluded authors for the multi-author task; wherein
the task configurator is operationally coupled to the exclusion list manager and excludes the excluded authors when assigning the multi-author task to users stored in the user database.

11. The workflow management system according to claim 10, wherein the users includes one of the following:
an individual; and
an assignment group including more than one individual; and
the multi-author task is a task assigned to at least a plurality of individuals and/or the assignment group.

12. The workflow management system according to claim 10, wherein the exclusion list manager:
adds an assignment group to the exclusion list when an individual in the assignment group performs the multi-author task;
adds an individual in an assignment group to the exclusion list when the individual performs the multi-author task with a request for review by co-authors of the assignment group; and
removes a user from the exclusion list when an individual performs the multi-author task with a request for review by the user listed in the exclusion list.

13. The workflow management system according to claim 10, wherein the task configurator:
assigns the multi-author task to users according to the rules of the workflow;
receives the exclusion list for the multi-author task from the exclusion list manager; and
filters the users to which the multi-author task is assigned using the exclusion list, thus preventing assigning the multi-author task to the excluded authors listed in the exclusion list.

14. The workflow management system according to claim 10, wherein the task configurator subsequently assigns the multi-author task whenever the multi-author task is performed by an author.

15. The workflow management system defined in claim 10, wherein the exclusion list manager includes:
an exclusion list generator that generates the exclusion list of excluded authors for the multi-author task; and
an exclusion list updater that:
adds an assignment group to the exclusion list when an individual in the assignment group performs the multi-author task;
adds an individual in an assignment group to the exclusion list when the individual performs the multi-author task with a request for review by users of the assignment group; and
removes a user from the exclusion list when an individual performs the multi-author task with a request for review by the user listed in the exclusion list.

16. A method for managing exclusion lists in the workflow management system defined in claim 10, the method comprising the steps of:
generating the exclusion list of excluded authors for the multi-author task; and
updating the exclusion list by:
adding an assignment group to the exclusion list when an individual in the assignment group performs the multi-author task;
adding an individual in an assignment group to the exclusion list when the individual performs the multi-author task with a request for review by users of the assignment group; and
removing a user from the exclusion list when an individual performs the multi-author task with a request for review by the user listed in the exclusion list.

17. The workflow management system defined in claim 10, wherein the task configurator includes:
a workflow interface that receives one of the workflows from the workflow database;
a user interface that receives the users from the user database;
a task assigner that assigns the multi-author task to the users according to the rules of the workflow;
an exclusion list interface that receives the exclusion list of excluded authors for the multi-author task;
a task filter that filters the users to which the multi-author task is assigned using the exclusion list, thus preventing assigning the multi-author task to the excluded authors.

18. A method for configuring tasks in the workflow management system defined in claim 10, the method comprising the steps of:
receiving the workflow from the workflow database;
receiving the users from the user database;
configuring tasks to produce the medical report according to the rules of the one of the workflows;
assigning the multi-author task to the users according to the rules of the workflow;
receiving the exclusion list of excluded authors for the multi-author task; and
filtering the users to which the multi-author task is assigned using the exclusion list, thus preventing assigning the multi-author task to the excluded authors.
